(19) United States
(12) Patent Application Publication
(10) Pub. No.: US 2004/0058457 A1
(43) Pub. Date: Mar. 25, 2004

(54) Functionalized nanoparticles

(76) Inventors: Xueying Huang, Hockessin, DE (US); Ming Zheng, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/630,262

(22) Filed: Jul. 30, 2003

Related U.S. Application Data
(60) Provisional application No. 60/406,736, filed on Aug. 29, 2002.

(51) Int. Cl. G01N 33/551; G01N 33/553

(52) U.S. Cl. 436/524; 436/525

(57) ABSTRACT

A functionalized nanoparticle is provided. The nanoparticle is comprised of a nanoparticle coated with a monolayer to which a bifunctional pepetide is attached. The peptide is functionalized to bind various biopolymers including DNA and RNA. The functionalized nanoparticle is useful in the capture of biopolymers and for the programmed assembly of nanometer scale electronic devices.

This application claims the benefit of U.S. Provisional Application No. 60/406,736, filed Aug. 29, 2002 the entire disclosure of which is hereby incorporated by reference.

### FIELD OF INVENTION

The invention relates to the field of nanotechnology. In particular the invention provides a functionalized nanoparticle having a peptide ligand which is functionalized to bind biopolymers for the assembly of nanodevices.

### BACKGROUND

Nanomaterials as hybrids with biological molecules have potential in electronic, optics, genomics, proteomics, and biomedical and bioanalytical applications. The ability to use biomolecules as templates in many of these applications depends on how well one can achieve rational design based on specific binding between inorganic nanomaterials and biological molecules. DNA is an attractive biomolecule to use as a template because of it's simplicity and specificity and it's ability to be manipulated.

The literature reports the use of complementary strands of DNA to assemble structures of metallic nanoparticles. Mirkin, C., et al., (Nature, Vol. 382, August 1996, pg. 607) prepared aggregates and Yun, C. et al. (JACS (2002), 124(26), 7644-7645) prepared dimers using gold nanoparticles labeled with oligonucleotides. Mucic, R., et al., (JACS, 1998, 120, pg 12674-5) also prepared aggregate, non-structured networks of gold nanoparticles using a short chain oligonucleotide as a bridge to complementary oligonucleotide ligands attached to the nanoparticles. Alivisatos, et al., (Nature, Vol. 382, August 1996, pg. 609) used a single-stranded DNA template to bind gold nanoparticles with the complementary sequences.

U.S. patent application No. 2002/0072069 describes a method for binding metallic nanoparticles to a nucleic acid molecules using a bifunctional linker wherein one binding region can be attached to the nucleic acid by the use of an intercalating, groove-binding, or alkylating agent.

The above functionalized nanoparticles are useful for addressing the problem of assembly of nanoparticle into devices however suffer from the drawback of being limited to DNA binding ligands where binding is dependant on nucleic acid hybridization. A functionalized nanoparticle having broader binding options would be useful in the industry.

One method of using DNA as a template to assemble nanoparticles would be to utilize a protein that has bifunctionality which would bind with high affinity and specificity to DNA as well as nanoparticles. One approach would be to create a fusion protein comprised of a DNA binding domain and a nanoparticle binding domain.

In spite of the attachment of other ligands to nanoparticles, there is no report of the use of a bifunctional peptide being used in conjunction with nanoparticles or other nanostructures for the generation of nano-scale conducting devices. A partial explanation for this deficiency in the art may be that there are significant difficulties in creating nanopartilces that are specifically functionalized with non-nucleic acid ligands.

Applicants have solved the deficiencies in the prior art by using a bifunctional protein or peptide that has one binding domain that can bind to a nanoparticle, and a second binding domain that can selectively bind to a biological template such as DNA. Such a nanoparticle can then be self-assembled into rational.

### SUMMARY OF THE INVENTION

The invention provides a nanoparticle useful for assembly into nanodevices. The nanoparticle is coated with a monolayer that is functionalized to recived a bi-functional protein. The bifucntional protein binds at one domain to the surface of the nanoparticle and at a second domain to a nucleic acid. Nanoparticles of the invention may be designed to be amenable to self assembly into nanodevices.

Accordingly in one embodiment the invention provides a functionalized nanoparticle comprising:

a) a nanoparticle coated with a monolayer comprising a capture coating component;

b) a bifunctional protein having a first binding domain and a second binding domain, the first and second binding domains each comprising a member of a binding pair;

wherein the bifunctional protein is affixed to the nanoparticle of (a) at the first binding domain.

In another embodiment the invention provides a method for capturing a capture moiety comprising contacting a capture moiety selected from the group consisting of nucleic acids, peptides, biological cells, and inorganic nanotubes with the functionalized nanoparticle of the invention. Preferred capture moieties are nucleic acids.

Additionally the invention provides a nucleic acid nanoparticle complex comprising:

a) a nanoparticle coated with a monolayer comprising a capture coating component;

b) a bifunctional protein having a first binding domain and a second binding domain, the first binding domain comprising a member of a binding pair, the second binding domain comprising a nucleic acid binding amino acid sequence;

wherein the bifunctional protein is affixed to the nanoparticle of (a) through the first binding domain and is affixed to a nucleic acid fragment at nucleic acid binding amino acid sequence.

In another embodiment the invention provides a process for immobilizing a multiplicity of nanaoparticles on a nucleic acid matrix comprising:

a) providing a multiplicity of functionalized nanoparticles, each comprising:

i) a nanoparticle coated with a monolayer comprising a capture coating component;

ii) a bifunctional protein having a first binding domain and a second binding domain, the first binding domain comprising a member of a binding pair, the second binding domain comprising a nucleic acid binding amino acid sequence;

wherein the bifunctional protein is affixed to the metallic nanoparticle through the first binding domain and wherein the nucleic acid binding amino acid sequence of each second binding domain is unique;

b) providing a nucleic acid matrix having peptide binding domains having affinity for the nucleic acid binding amino acid sequence of each second binding domain of each bifunctional peptide of (a);

c) contacting the functionalized nanoparticles of (a) with the nucleic acid matrix of (b) under conditions whereby the nucleic acid binding amino acid sequence of the bifunctional peptide bind to peptide binding domains of the nucleic acid matrix of (b) to immobilize the nanoparticles.

Additionally the invention provides a nanometer scale electronic device made by the process of the invention.

In another embodiment the invention provides a functionalized carbon nanotube comprising:

a) a carbon nanotube; and

b) a bifunctional protein having a first binding domain and a second binding domain, the first binding domain having affinity for a carbon nanotube and the second binding domain comprising a member of a binding pair;

wherein the bifunctional protein is affixed to the carbon nanotube of (a) through the first binding domain.

### BRIEF DESCRIPTION OF THE DRAWINGS, AND SEQUENCE DESCRIPTIONS

FIG. 1 illustrates a gel showing over-expression and purification of the GST-Zif268 protein.

FIG. 2 is a gel shift assay of DNA binding by purified GST-Zif268 fusion protein.

FIG. 3 illustrates a nanoparticle binding assay on the purified GST-Zif268 fusion protein vs. bovine serum albumin (BSA) protein.

FIGS. 4A and 4B are gel shift assays on the purified GST-Zif268 protein binding to both Au-GSH nanoparticles and DNA. 4A is a gel image recorded with ScanJet 6300C. 4B is a gel image under UV illumination.

The invention can be more fully understood from the following detailed description and the accompanying sequence descriptions which form a part of this application.

The following sequences conform with 37 C. F. R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ. ID NO:1 is a DNA sequence of the zinc finger target sequence Zif268

SEQ. ID NO:2 is a DNA sequence of the zinc finger target sequence NRE

SEQ. ID NO:3 is a DNA sequence of the zinc finger target sequence Sp1C

SEQ. ID NOs:4-27 are amino acid sequences of the carbon nanotube binding proteins CNP1-CNP24.

SEQ. ID NO:28 is a amino acid sequence of the mouse zinc finger sequence Zif268

SEQ. ID NO:29 is a DNA sequence of the zinc finger target sequence Zif268

SEQ. ID NO:30 is a DNA sequence for the primer 821-ZNF1

SEQ. ID NO:31 is a DNA sequence for the primer 822-ZNF2

SEQ. ID NO:32 is a DNA sequence for the primer 823-ZNF3

SEQ. ID NO:33 is a DNA sequence for the primer 824-ZNF4

SEQ. ID NO:34 is a DNA sequence for the primer 825-ZNF5

SEQ. ID NO:35 is a DNA sequence for the primer 826-ZNF6

SEQ. ID NO:36 is a DNA sequence for the primer 827-ZNF7

SEQ. ID NO:37 is a DNA sequence for the primer 828-ZNF8

SEQ. ID NO:38 is a DNA sequence for the primer 829-ZNF9

SEQ. ID NO:39 is a DNA sequence for the primer 848-ZNR10

SEQ. ID NO:40 is a DNA sequence for the primer 849-ZNR11

SEQ. ID NO:41 is a DNA sequence for the primer 850-ZNR12

SEQ. ID NO:42 is a DNA sequence for the primer 851-ZNR13

SEQ. ID NO:43 is a DNA sequence for the primer 852ZNR14

SEQ. ID NO:44 is a DNA sequence for the primer 853-ZNR15

SEQ. ID NO:45 is a DNA sequence for the primer 854-ZNR16

SEQ. ID NO:46 is a DNA sequence for the primer 855-ZNR17

SEQ. ID NO:47 is a DNA sequence for the primer 856-ZNR18

SEQ. ID NO:48 is a DNA sequence for the primer 897-2ZnRE

SEQ. ID NO:49 is a DNA sequence for the primer 872-NZF

SEQ. ID NO:50 is a DNA sequence for the primer 873-NZR

SEQ. ID NO:51 is a DNA sequence for the primer 874-N/ZF

SEQ. ID NO:52 is a DNA sequence for the primer 875-N/ZR

SEQ. ID NO:53 is a DNA sequence for the primer 876-N//ZF

SEQ. ID NO:54 is a DNA sequence for the primer 877-N//ZR

SEQ. ID NO:55 is a DNA sequence for the primer 878-CAPB5′

SEQ. ID NO:56 is a DNA sequence for the primer 879-CAPB3′

SEQ. ID NO:57 is a DNA sequence for the primer 880-CAPE5′

SEQ. ID NO:58 is a DNA sequence for the primer 881-CAPE3′

### DETAILED DESCRIPTION OF THE INVENTION

The invention provides a functionalized nanoparticle comprising a bifunctional peptide ligand affixed to the surface. The peptide is functionalized at the portion distill to the nanoparticle to bind to a variety of materials including biopolymers such as nucleic acids, nanotubes and other peptides.

The functionalized nanoparticles may be immobilized on matrices of biopolymers to form an electrical conducting network that will serve as the foundation for nano-circuitry. Immobilized nanoparticles have utility in the field of nanoscale electronic devices, multifunctional catalysts, chemical sensors, and many biological applications such as biosensors, biological assays.

A number of abbreviations and definitions are used throughout this disclosure and may be used for the interpretation of the claims and the specification.

“CNBP” means Carbon nanotube binding peptide

“MWNT” means Multi-walled nanotube

“SWNT” means Single walled nanotube

“TEM” means transmission electron microscopy

“CNT” means carbon nanotube

“GSH” refers to the chemical compound glutathione.

“TP” is the abbreviation for tiopronin

“HTH is the abbreviation for helix-turn-helix

“Nanoparticles” are herein defined as metallic or semiconductor particles with an average particle diameter of between 1 and 100 nm. Preferably, the average particle diameter of the particles is between about 1 and 40 nm. As used herein, “particle size” and “particle diameter” have the same meaning.

An “alloy” is herein defined as a homogeneous mixture of two or more metals.

A “monolayer” refers to a layer of material coated on a nanoparticle that is the thickness of single molecule.

A “mixed monolayer” refers to a monolayer having at least two different molecular components.

A “capture coating component” as used herein refers to a material capable of forming a monolayer on a nanoparticle that has an affinity for some ligand or capture moiety. The “capture” component makes up the lesser portion of a mixed monolayer and may comprise less than 50% of the monolayer.

A “shielding coating component” refers to a material capable of forming a monolayer on a nanoparticle that has the ability to prevent non-specific binding of substances that are not capture moieties. Shielding coating components may be comprised of a variety of materials where ethylene glycol is particularly suitable.

The term “peptide” refers to two or more amino acids joined to each other by peptide bonds or modified peptide bonds. Peptides include those modified either by natural processes, such as processing and other post-translational modifications, but also chemical modification techniques. The modifications can occur anywhere in a peptide, including the peptide backbone, the amino acid side chain, and the amino or carboxyl terminal. Examples of modifications include but are not limited to amidation, acylation, acetylation, cross linking, cyclization, glycosylation, hydroxylation, phosphorylation, racemization, and covalent attachment of various moieties such as nucleotide or nucleotide derivative, lipid or lipid derivatives (see, for instance, Proteins-Structure and Molecular Properties, 2^{nd} Ed Creighton, W. H. Freeman and Company, New York (1993) and Post-translation covalent Modification of Proteins, B. C. Johnson, Ed., Academic Press, New York (1983)).

The term “amino acid” will refer to the basic chemical structural unit of a protein or polypeptide. The following abbreviations will be used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamine | Gin | Q |
| Glutamine acid | Glu | E |
| Glutamine or glutamic acid | Glx | Z |
| Glycine | Gly | G |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “peptide” and “polypeptide” will be used interchangeably.

As used herein the term “bifunctional” as it refers to a peptide will mean a peptide that possess affinity for at least two different entities. Typical bifunctional peptides of the invention are those that are functionalized at the C-terminus or N-terminus to bind to a nanoparticle whereas the opposite terminus is functionalized to bind to some capture moiety, typically a biopolymer or nanostructure such as a carbon nanotube.

The term “binding domain” refers to portions of a bifunctional peptide that have binding affinity for a particular entity or capture moiety,

The term “carbon nanostructure binding peptide” refers to peptides that were selected to bind with a carbon nanostructures. Where peptides are generated with specific affinity to carbon nanotubes, these peptides will be referred to as “carbon nanostructure binding peptides” or CNBP's.

The term “nano-structure” means tubes, rods, cylinders, bundles, wafers, disks, sheets, plates, planes, cones, slivers, granules, ellipsoids, wedges, polymeric fibers, natural fibers, and other such objects which have at least one characteristic dimension less than about 100 microns.

The term “nano-rod” means a variety of nano-structures which may be either hollow or solid and may or may not have a circular corssectional shape. Nano-rods of the invention may include nanotubes, nanofibers, polymeric nanofibers, bundles and multiwalled structures.

The term “nanotube” is one subset of a nano-rod and refers to a hollow article having a narrow dimension (diameter) of about 1-200 nm and a long dimension (length), where the ratio of the long dimension to the narrow dimension, i.e., the aspect ratio, is at least 5. In general, the aspect ratio is between 10 and 2000.

By “nanoplanes” is meant surfaces which have one characteristic dimension less than 500 nanometer, for example a single or a dual layer of graphite or graphene sheets.

By “nanofibers” is meant natural or polymeric filaments which have a small dimension of less than 1000 nanometer.

By “carbon-based nanotubes” or “carbon nanotube” herein is meant hollow structures composed primarily of carbon atoms. The carbon nanotube can be doped with other elements, e.g., metals.

The term “functionalized carbon nanotube” refers to a carbon nanotube having a bifuntional protein or peptide attached thereto. Functionalized carbon nanotubes of the invention will posses bifunctional peptides affixed to the nanotube and having a free binding domain that will have affinity for a capture moiety such as a nucleic acid molecule.

The term “binding pair” refers to chemical or biopolymer based couples that bind specifically to each other. Common examples of binding pairs are immune-type binding pairs, such as antigen/antibody or hapten/anti-hapten systems.

As used herein the term “solid support” refers to a material on which a nucleic acid matrix may be immobilized, e.g. nitrocellulose or nylon.

As used herein, a “nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

The term “nucleic acid binding amino acid sequence” refers to a stretch of contiguous amino acids which have an affinity for specific nucleic acid target sequences.

The term “target sequence” means a stretch of nucleic acids which are specifically recognized and bound to a nucleic acid binding peptide.

The terms “zinc-finger” or “Zn-finger” refers to a specific amino acid sequence that binds a specific nucleic acid sequence. Zn-finger sequences are well known in the art and have specificity for target stretches of DNA including but not limited to: (i) GCGTGGGCG [SEQ ID NO:1] also referred to herein as Zif268 (Kim et al, Proc Natl Acad Sci U S A. Mar. 17, 1998;95(6):2812-7); (ii) AAGGGTTCA [SEQ ID NO:2], also referred to herein as NRE, (Kim et al., supra); and GGGGCGGGG [SEQ ID NO:3], also referred to herein as Sp1C, (Wolfe et al., Annu Rev Biophys Biomol Struct. 2000;29:183-212.)

A “capture moiety” refers to a substance that may be bound by a binding domain on a bifunctional peptide. Capture moieties may be any substance but are typically the biopolymers such as peptides, proteins and nucleic acid fragments but may also include a variety of nanostructures.

The term “phage” or “bacteriophage” refers to a virus that infects bacteria. Altered forms may be used for the purpose of the present invention. The preferred bacteriophages are derived from two “wild” phages, called M13 and lambda. Lambda phages are used to clone segments of DNA in the range of around 10-20 kb. They are lytic phages, i.e., they replicate by lysing their host cell and releasing more phages. The M13 system can grow inside a bacterium, so that it does not destroy the cell it infects but causes it to make new phages continuously. It is a single-stranded DNA phage.

The term “phage display” refers to the display of functional foreign peptides or small proteins on the surface of bacteriophage or phagemid particles. Genetically engineered phage could be used to present peptides as segments of their native surface proteins. Peptide libraries may be produced by populations of phage with different gene sequences.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature.

“Coding sequence” of “coding region” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from the nucleic acid fragment of the invention. Expression may also refer to translation of mRNA into a polypeptide.

The term “signal peptide” refers to an amino terminal polypeptide preceding the secreted mature protein. The signal peptide is cleaved from and is therefore not present in the mature protein. Signal peptides have the function of directing and translocating secreted proteins across cell membranes. Signal peptide is also referred to as signal protein.

The terms “plasmid”, “vector” and “cassefte” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “heterojunction” will refer the juxtapostion of at least two different materials in an ordered fashion in a defined space.

Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987).

Coated Nanoparticies

The invention provides a nanoparticle coated with a monolayer that serves as a point of attachment for a bifunctional peptide. Suitable nanoparticles are metallic or semiconductor particles with an average particle diameter of between 1 and 100 nm. Preferably, the average diameter of the particles is between about 1 and 40 nm. The metallic nanoparticles include, but are not limited to, particles of gold, silver, platinum, palladium, iridium, rhodium, osmium, iron, copper, cobalt, and alloys composed of these metals. The “semiconductor nanoparticles” include, but are not limited to, particles of cadmium selenide, cadmium sulfide, silver sulfide, cadmium sulfide, zinc sulfide, zinc selenide, lead sulfide, gallium arsenide, silicon, tin oxide, iron oxide, and indium phosphide.

The nanoparticles of the invention are coated with a monolayer. The monolayer serves as an attachment for a bifunctional peptide, and also serves to render the nanoparticle water soluble. Nanoparticles that have been rendered water soluble by coating are referred to herein as “stabilized”. Methods for the preparation of stabilized, water-soluble metal and semiconductor nanoparticles are known in the art. These particles can be either charged or neutral depending on the nature of the organic coating. For example, Templeton et al. (Langmuir 15:66-76 (1999)), herein incorporated by reference, describe a method for the preparation of stabilized, charged, water-soluble gold nanoparticles protected by tiopronin or coenzyme A monolayers. To prepare the tiopronin-protected gold nanoparticles, tetrachloroauric acid and N-(2-mercaptopropionyl)glycine (tiopronin) were codissolved in a mixture of methanol and acetic acid. Sodium borohydride was added with rapid stirring. The average particle size of these particles could be controlled by varying the mole ratio of tiopronin to tetrachloroauric acid in the reaction. The coenzyme A protected gold nanoparticles were prepared in a similar manner by substituting coenzyme A for tiopronin in the reaction.

A similar method of preparing stabilized, water-soluble nanoparticles of the metals gold, silver, platinum, palladium, cobalt and nickel is descried by Heath et al. in U.S. Pat. No. 6,103,868, herein incorporated by reference. In this method, a solution or dispersion of one or more metal salts was mixed with a solution of an organic surface passivant that had a functional group such as a thiol, phosphine, disulfide, amine, oxide, or amide. A reducing agent was then added to reduce the metal salt to the free metal.

Other suitable methods for the preparation of coated nanoparticles are known (see for example Chen et al. (Colloids and Surfaces A 169:107-116 (2000)); Hagemeyer et al. in U.S. Pat. No. 6,074,979; Wuelfing et al. (J. Am. Chem. Soc. 120:12696-12697 (1998)); Chan et al. (Science 281:2016-2018 (1998)); Mitchell et al. (J. Am. Chem. Soc. 121:8122-8123 (1999)); and Napper (J. Colloid. Interface. Sci 58:390-407 (1977)).

For both stabilized, water-soluble semiconductor and metal nanoparticles it is possible to use mixtures of various stabilizing coatings or monolayers, for example, poly(ethylene glycol) and glutathione or poly(ethylene glycol) and tiopronin.

Thus it is within the scope of the invention to provide functionalized nanoparticle comprising:

a) a nanoparticle coated with a monolayer comprising a capture coating component;

b) a bifunctional protein having a first binding domain and a second binding domain, the first and second binding domains each comprising a member of a binding pair;

wherein the bifunctional protein is affixed to the nanoparticle of (a) at the first binding domain.

Alternatively it may be useful to provide a nanoparticle coated with a mixed monolayer where one component of the monolayer provides a point of attachment for the bifunctional peptide, and the other component of the mixed monolayer acts as a shield against non-specific binding of undesired proteins or capture moieties. In this embodiment, typically the mixed monolayer having a capture component and a shielding component are part of the same monolayer. Typically the capture component comprises less than about 50% of the mixed monolayer where about 20%-40% is preferred. Conversely the shielding component forms the major component of the monolayer and comprises at least about 50% of the monolayer, where 60% to about 90% is preferred.

The capture component of any such mixed monolayer must have the ability to bind the bifuctional peptides of the invention. The capture component may be functionalized with various chemical groups that allow for binding to a capture moiety. Non-limiting examples of such chemical reactive groups include those selected from the group consisting of: —NH₂, —COOH, —CHO—, —OH, —X (Cl, Br, I), succinimide, and epoxy groups. Preferred examples of suitable capture components are tiopronin and GSH. Tiopronin (abbreviated TP), is N-2-mercaptopropionyl-glycine is particularly suitable as a capture component because of the presence of exposed carboxy groups which serves as a convenient binding site for peptides.

The shielding component of the mixed monolayer serves to block the binding of non-capture moiety materials to the coated nanoparticle and permits the nanoparticle to be used to bind, isolate or immobilize specific bifunctional proteins or capture moieties. Suitable shielding components will include but are not limited to short chain ethylene glycol oligomers, ethylene glycol methacrylate, sugars, crown ethers, and acrylamide, where the short chain ethylene glycol oligomers are preferred.

Bifunctional Peptide

The invention provides a bifunctional peptide that is affixed on the stabilized coated nanoparticle. The bifunctional peptide will typically have at least two binding domains. One binding domain will have affinity for the monolayer coating the nanoparticle. The other binding domain will have affinity for some capture moiety.

Bifunctional peptides of the invention will be from about 5 to about 100-amino acids in length where about 10 to about 30 amino acids is preferred and will generally attain a linear secondary structure.

The bifunctional peptide will be engineered to have at least one binding domain that has affinity for the monolayer coating the nanoparticle. Typically this binding domain will be a member of a binding pair. Chemical and protein based binding pairs are well known in the art and include, but are not limited to combinations such as, Glutathione-S-transferase/glutathione, 6× Histidine Tag/Ni-NTA, Streptavidin/biotin, S-protein/S-peptide, Cutinase/phosphonate inhibitor. Additionally binding pairs will include any of the class of immune-type binding pairs, such as antigen/antibody or hapten/anti-hapten systems; and also any of the class of nonimmune-type binding pairs, such as biotin/avidin; biotin/streptavidin; folic acid/folate binding protein; complementary nucleic acid segments, including peptide nucleic acid sequences; protein A or G/immunoglobulins; and binding pairs, which form covalent bonds, such as sulfhydryl reactive groups including maleimides and haloacetyl derivatives, and amine reactive groups such as isotriocyanates, succinimidyl esters and sulfonyl halides.

In another aspect the bifunctional peptide will be designed to include a second binding domain that has affinity for a capture moiety. Typical capture moieties of the invention include for example, biopolymers such as peptides, proteins, nucleic acid fragments, and collagen, as well as nanomaterials useful in the assemble and synthesis of nanodevices such as for example various nano-structures (nano-rods, nano-tubes, nano-planes and nano-fibers as defined herein). Preferred in the present invention are nucleic acid and carbon nanotube capture moieties.

Nucleic acid binding domains or motifs are well known in the art and many are suitable for use in the present invention. For example, one common family is distinguished by the “helix-turn-helix” motif. These sequences include without limitation: E. coli CRP (Genbank accession number: NP_{—}417816) (McKay, D. B. and Steitz, T. A. 1981, Nature, 290,744-749), λcro(Genbank accession number: P03040) (Anderson et al., 1981, Nature, 290, 754-758.), and λcl (Genbank accession number: NP_{—}040628) (Pabo, C. O. and Lewis, M., 1982, Nature, 298, 443447). All three of these examples are dimers of relatively small polypeptides (=100 aa in length), composed of a surface exposed pair of alpha helices separated by an unusual tight turn of polypeptide. The spatial separation of these helix-turn-helix (HTH) motifs in each dimer is essentially identical and equal to 34 Å, which is also the relative separation of consecutive major grooves of B-form DNA along one face of a duplex. Protein sequence database searches have identified hundreds of HTH containing DNA binding proteins from both prokaryotic and eukaryotic cells (Harrison, S. C. ,1991, Nature, 353, 715-719; Aggarwaal, A. K. and Harrison, S.C., 1990, Annu. Rev. Biochem., 59, 933-969).

Other types of DNA binding domains are also suitable for use in the invention. Over 150 of proteins of non “HTH” type have been studied at the molecular level in complex with their DNA targets, providing a large database for this particular type of intermolecular recognition event (Lilley, D. M. J. (ed.), 1995, DNA-protein structural interactions. IRL Press, OUP, Oxford; Harrison, S.C., 1991, Nature, 353,715-719). Many of these other motifs also contain a recognition alpha helix, as in the helix-loop-helix structures that are common in eukaryotic transcription factors, where the constraints on the region between helices of the HTH are relaxed, and in zinc fingers (Luisi, B. F., 1992, Nature, 356, 379-380). A distinct recognition motif based on a pair of polypeptide strands in an anti-parallel β-ribbon has also been identified in the phage P22 repressors Arc (Genbank accession number: NP_{—}059642) and Mnt (Genbank accession number: NP_{—}059641), the E. coli methionine repressor protein, Motif (Genbank accession number: NP_{—}418373), and in the bacterial DNA-packaging protein HU (Raumann et al., 1994, Curr. Op. Struct.Biol., 4, 36-43). The eukaryotic TATA binding protein also uses a β-sheet recognition motif (Patikoglou, G. and Burley, S. K., 1997, Annu. Rev. Biophys. Biomol. Struct., 26, 289-325).

Additionally RNA binding domains or motifs may also be used. Crystal structures are available for three tRNA synthetase (TRS) complexes (Cusack, S., 1995, Nature Struct. Biol., 2, 824-831.), the MS2 bacteriophage translational repression complex (Valegard et al., 1994, Nature, 371, 623-626; Valegard et al., 1997, J. Mol. Biol., 271, 724-738), and the U1A complex from the spliceosome (Oubridge et al., 1994, Nature, 372, 432-438; Nagai, K., 1996, Curr Op. Struct. Biol., 6, 53-61). Common target sites for proteins are stern-loops, stern-bubbles, pseudo-knots, and complex sites made up of such smaller sites. Protein motifs which recognize such sites include viral coat protein subunits (Rossmann, M. G. and Johnson, J. E., 1989, Annu. Rev. Biochem., 58, 533-573; Valegard et al. 1990, Nature, 345, 36-41); a conserved RNA-binding domain(RBD) of 90-100 amino acids in length (St. Johnston et al., 1992, PNAS (USA), 89, 10979-10983; Bycroft et al., 1995, EMBO J., 14,3563-3571; Kharrat etaL, 1995, EMBO J., 14, 3572-3584); proteins containing the RGG box, a 20-25 amino acid region containing several arginine-glycine-glycine sequence repeats interspersed with other, often aromatic, residues; the k homology (KH) domain of ˜50 amino acids in length containing the octapeptide, IGX2GXJ (where X is any amino acid); and the so-called arginine fork (Nagai, K., 1996, Curr Op. Struct. Biol., 6, 53-61). The most common RNA-binding domain is the RNP domain, which occurs in at least 600 proteins in higher organisms (Musco et al., 1996, Cell, 85, 237-245; Nagai et al., 1990, Nature, 348, 515-520; Avis et al., 1996, J. Mol. Biol., 257,398-411).

Preferred in the present invention are the Zn-finger binding proteins which exhibit high binding affinity and specificity for target sequences, and which are readily available and described in the literature. Preferred Zn finger proteins for use in the present invention are those that bind to the following target sequences: GCGTGGGCG [SEQ ID NO:1]; AAGGGTTCA [SEQ ID NO:2] and GGGGCGGGG [SEQ ID NO:3].

Alternatively the bifunctional protein of the invention may be designed to bind various nano-structures such as nanorods, nanofibers or nanotubes. It is possible to generate peptides that will bind to carbon nanotubes for example. Nanotube binding peptides of the may be generated randomly and then selected against a population of carbon nanostructures for binding affinity to CNT's. The generation of random libraries of libraries of peptides is well known and may be accomplished by a variety of techniques including, bacterial display (Kemp, D. J.; Proc. Natl. Acad. Sci. USA 78(7): 4520-4524, 1981, and Helfman, D. M., et al., Proc. Natl.Acad. Sci. USA 80(1): 31-35, 1983) yeast display (Chien Conn., et al., Proc Natl Acad Sci USA 1991 Nov 1; 88(21): 9578-82.) combinatorial solid phase peptide synthesis (U.S. Pat. No. 5,449,754, U.S. Pat. No. 5,480,971, U.S. Pat. No. 5,585,275, U.S. Pat. No. 5,639,603) and phage display technology (U.S. Pat. No. 5,223,409; U.S. Pat. No. 5,403,484; U.S. Pat. No. 5,571,698; U.S. Pat. No. 5,837,500). Techniques to generate such biological peptide libraries are described in Dani, M., J. of Receptor & Signal Transduction Res., 21(4), 447-468 (2001).

A preferred method to randomly generate peptides is by phage display. Phage display is an in vitro selection technique in which a peptide or protein is genetically fused to a coat protein of a bacteriophage, resulting in display of fused protein on the exterior of phage viron, while the DNA encoding the fusion residues within the virion. This physical linkage between the displayed protein and the DNA encoding it allows screening of vast numbers of variants of proteins, each linked to a corresponding DNA sequence, by a simple in vitro selection procedure called “biopanning.” In its simplest form, biopanning is carried out by incubating the pool of phage-displayed variants with a target of interest that has been immobilized on a plate or bead, washing away unbound phage, and eluting specifically bound phage by disrupting the binding interactions between the phage and target. The eluted phage is then amplified in vivo and the process repeated, resulting in stepwise enrichment of the phage pool in favor of the tightest binding sequences. After 3 or more rounds of selection/amplification, individual clones are characterized by DNA sequencing.

Using the above methods applicants have generated a number of peptides that selectively bind carbon nanotubes, listed below:

| | SEQ ID | |
|---|---|---|
| CNP | NO: | Sequence |
| CNP1 | SEQ. ID NO.: 4 | DPHHHWYHMHQH |
| CNP2 | SEQ. ID NO.: 5 | HAHSQWWHLPYR |
| CNP3 | SEQ. ID NO.: 6 | HAHSRRGHIQHR |
| CNP4 | SEQ. ID NO.: 7 | HCHHPWGAWHTL |
| CNP5 | SEQ. ID NO.: 8 | HCWNQWCSRHQT |
| CNP6 | SEQ. ID NO.: 9 | HGNWSYWWSKPS |
| CNP7 | SEQ. ID NO.: 10 | HHWHHWCMPHKT |
| CNP8 | SEQ. ID NO.: 11 | HNWYHWWMPHNT |
| CNP9 | SEQ. ID NO.: 12 | HNWYRWCIRHNN |
| CNP10 | SEQ. ID NO.: 13 | HRWYRWSSRNQT |
| CNP11 | SEQ. ID NO.: 14 | HSSWWLALAKPT |
| CNP12 | SEQ. ID NO.: 15 | HWCAWWISSNQS |
| CNP13 | SEQ. ID NO.: 16 | HWKHPWGAWDTL |
| CNP14 | SEQ. ID NO.: 17 | HWSAWWIRSNQS |
| CNP15 | SEQ. ID NO.: 18 | HWSPWHRPWYQP |
| CNP16 | SEQ. ID NO.: 19 | HYSWYSTWWPPV |
| CNP17 | SEQ. ID NO.: 20 | HYWWRWWMPNQT |
| CNP18 | SEQ. ID NO.: 21 | KCHSRHDHIHHH |
| CNP19 | SEQ. ID NO.: 22 | KSLSRHDHIHHH |
| CNP20 | SEQ. ID NO.: 23 | KSRSRHDEIHHH |
| CNP21 | SEQ. ID NO.: 24 | KYRSRHDHIHHH |
| CNP22 | SEQ. ID NO.: 25 | QWHSRHDHIHHH |
| CNP23 | SEQ. ID NO.: 26 | HHHHLRHPFWTH |
| CNP24 | SEQ. ID NO.: 27 | WPHHPHAAHTIR |

It is contemplated that these peptides, or any other similarly generated may be incorporated into a bifunctional peptides for the purpose of binding carbon nanotubes.

Once a design has been established for the bifunctional peptide, the peptide may be synthesized and produced by a variety of means well known in the art. For example, the peptide may be synthesized by solid state peptide synthesis where amino acids are sequentially added on a resin and then cleaved off the resin for purification (see for example, Yokum et al., Solid-Phase Synthesis (2000), 79-102. Editor(s): Kates, Steven A.; Albericio, Fernando. Publisher: Marcel Dekker, Inc., New York, N.Y.; Albericio, Fernando, Biopolymers (2000), 55(2), 123-139; Fields, Gregg B. Molecular Biomethods Handbook (1998), 527-545. Editor(s): Rapley, Ralph; Walker, John M. Publisher: Humana, Totowa, N.J.) Alternatively the peptide may be produced by recombinant means. DNA coding regions may be designed and chemically synthesized that encode for the peptide of choice. These fragments may then be cloned into cloning and expression vectors that are suitable for the transformation of various microbial expression hosts.

Microbial expression systems and expression vectors containing regulatory sequences, such as promoters, that direct high level expression of foreign proteins are well known to those skilled in the art. Any of these could be used to construct chimeric genes for production of the any of the gene products of the instant sequences.

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host.

Initiation control regions or promoters, which are useful to drive expression of theses genes in the desired host cell are numerous and familiar to those skilled in the art. Virtually any promoter capable of driving these genes is suitable for the present invention including but not limited to CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, ara, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

Optionally it may be desired to produce the instant gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the host production host. Methods for choosing appropriate signal sequences are well known in the art (see for example EP 546049;WO 9324631). The secretion signal DNA or facilitator may be located between the expression-controlling DNA and the instant gene or gene fragment, and in the same reading frame with the latter.

Linkinq Bifunctional Peptides to Coated Nanaoparticle

The bifunctional peptide can be affixed to the coated nanoparticles by a variety of methods. These can be divided into two categories according to the chemical nature of linkage: covalent and non-covalent. The covalent linking method employs a small cross-linker molecule to react with a functional group on the coated nanoparticles and one on the bifunctional peptide. The basic principle of and many cross-linker molecules for the method are well-described in the literature (Bioconiuqate Techniques by Greg T. Hermanson. Academic Press, San Diego, Calif.,1996). One example of this method uses molecule EDC [1-ethyl-3-(3-dimethylaminopropyl)carbodiimide hydrochloride] to cross-link a carboxylic group on the coated nanoparticle (Au-Tp, for instance)) and a amine group on the bifunctional peptide.

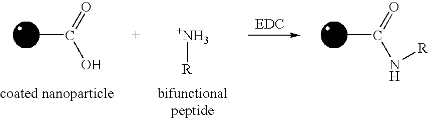

The non-covalent method employs a non-covalent interaction between a ligand pre-affixed on the coated nanoparticle (Au-GSH, for example) and the corresponding ligand-binding domain (GST for Au-GSH) of the bifunctional peptide. A variety of ligand/ligand binding domain pairs can be used for the purpose, as described in previous text.

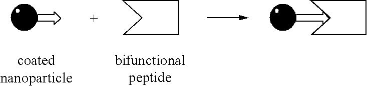

Immobilized Nanoparticles

Nanoparticles having an attached bifunctional protein or peptide may be immobilized through one of the binding domains of the peptide to a variety of capture moieties. Typically the capture moiety will conform to a specific structure and may itself be immobilized on a solid support. Typical capture moieties of the invention include for example, biopolymers such as peptides, proteins, nucleic acid fragments, and collagen, as well as nanomaterials useful in the assemble and synthesis of nanodevices such as for example various nano-structures (nano-rods, nano-tubes, nano-planes and nano-fibers as defined herein).

One of the advantages of a CNT binding peptide in the context of the invention is that it allows for the use of the CNT in place of the typical nanoparticle. So, for example a CNT binding peptide, functionalized with a nucleic acid binding domain would be useful to link a CNT and a nucleic acid molecule or a nucleic acid matrix. Such a design simplifies the need for the construction of a complex nanoparticle and allows for the construction of a matrix of CNT's according to the pattern established by the nucleic acid matrix.

Carbon nano-structures suitable as capture moieties or in the place of a nanoparticle are those structures comprised at primarily of carbon which take the form of tubes, rods, cylinders, bundles, wafers, disks, sheets, plates, planes, cones, slivers, granules, ellipsoids, wedges, polymeric fibers, natural fibers, and other such objects which have at least one characteristic dimension less than about 100 nm. Preferred carbon nano-structures of the invention are nanotubes.

Particularly suitable nanotubes are generally about 1-200 nm in diameter where the ratio of the length dimension to the narrow dimension, i.e., the aspect ratio, is at least 5. In general, the aspect ratio is between 10 and 2000. Carbon nanotubes are comprised primarily of carbon atoms, however may be doped with other elements, e.g., metals. The carbon-based nanotubes can be either multi-walled nanotubes (MWNTs) or single-walled nanotubes (SWNTs). A MWNT, for example, includes several concentric nanotubes each having a different diameter. Thus, the smallest diameter tube is encapsulated by a larger diameter tube, which in turn, is encapsulated by another larger diameter nanotube. A SWNT, on the other hand, includes only one nanotube.

Carbon nanotubes (CNT) may be produced by a variety of methods, and are additionally commercially available. Methods of CNT synthesis include laser vaporization of graphite (A. Thess et al. Science 273, 483 (1996)), arc discharge (C. Journet et al., Nature 388, 756 (1997)) and HiPCo (high pressure carbon monoxide) process (P. Nikolaev et al. Chem. Phys. Lett. 313, 91-97 (1999)). Chemical vapor deposition (CVD) can also be used in producing carbon nanotubes (J. Kong et al. Chem. Phys. Lett. 292, 567-574 (1998); J. Kong et al. Nature 395, 878-879 (1998); A. Cassell et al. J. Phys. Chem. 103, 6484-6492 (1999); H. Dai et al. J. Phys. Chem. 103,11246-11255 (1999)).

Alternatively the capture moiety may be a biopolymer such as a nucleic acid matrix or a peptides or population of peptides having a specific structural conformation. Where the bifunctional peptide of the invention has been functionalized to comprise a nucleic acid binding domain, it is preferred if the capture moiety is a nucleic acid.

The capture moiety may be immobilized on a solid support for the capture and subsequent immobilization of the nanoparticle through the bifunctional peptide. For this purpose suitable solid supports will include but are not limited to silicon wafers, synthetic polymer supports, such as polystyrene, polypropylene, polyglycidylmethacrylate, substituted polystyrene (e.g., aminated or carboxylated polystyrene; polyacrylamides; polyamides; polyvinylchlorides, etc.); glass, agarose, nitrocellulose, and nylon.

One of the important advantages of the invention is the aspect of programmability that is introduced into the construction of nanostructures through the design of the functionalized nanoparticle. It is contemplated that a multiplicity of functionalized nanoparticles may be constructed each comprising a bifunctional peptide having a specific nucleic acid binding domain. Each nucleic acid binding region will have a specific affinity for a specific stretch of nucleic acids. Thus, functionalized nanoparticles can be designed bind to specific regions of a nucleic acid matrix and their deposition can be precisely controlled. The ability to deposit metals or semiconductors in a precise and controlled fashion satisfies a critical need in the construction of nanometer scale electronic devices such as electronic heterojunctions, interconnects and nanocircuits.

It is thus within the scope of the invention to provide a nucleic acid nanoparticle complex comprising:

a) a nanoparticle coated with a monolayer comprising a capture coating component;

b) a bifunctional protein having a first binding domain and a second binding domain, the first binding domain comprising a member of a binding pair, the second binding domain comprising a nucleic acid binding amino acid sequence;

wherein the bifunctional protein is affixed to the nanoparticle of (a) through the first binding domain and is affixed to a nucleic acid fragment at nucleic acid binding amino acid sequence.

Additionally it is within the scope of the invention to provide a method for immobilizing a multiplicity of nanaoparticles on a nucleic acid matrix comprising:

a) providing a multiplicity of functionalized nanoparticles, each comprising:

i) a nanoparticle coated with a monolayer comprising a capture coating component;

ii) a bifunctional protein having a first binding domain and a second binding domain, the first binding domain comprising a member of a binding pair, the second binding domain comprising a nucleic acid binding amino acid sequence; wherein the bifunctional protein is affixed to the metallic nanoparticle through the first binding domain and wherein the nucleic acid binding amino acid sequence of each second binding domain is unique;

b) providing a nucleic acid matrix having peptide binding domains having affinity for the nucleic acid binding amino acid sequence of each second binding domain of each bifunctional peptide of (a);

c) contacting the functionalized nanoparticles of (a) with the nucleic acid matrix of (b) under conditions whereby the nucleic acid binding amino acid sequence of the bifunctional peptide bind to peptide binding domains of the nucleic acid matrix of (b) to immobilize the nanoparticles.

Nanometer-scale Electronic Device

The metallic or semiconductor nanoparticles of the invention, immobilized on a capture moiety matrix may be used in the construction of conductive heterojunctions and interconnects at the nano scale. In the metallic case, the immobilized nanoparticles are expected to be able to link nanometer scale electronic devices together permitting the fabrication of high density electronic circuits. It is contemplated that it will be possible to array the metallic nanoparticles in an arrangement, where the distance between adjacent nanoparticle can be controlled by the potential difference between them, then the matrix could be used as a non-volatile memory device similar to that proposed by Leiber and collaborators (Rueckes T. et al. (2000). Science 289, 94-97) for carbon nanotubes.

Semiconducting nanoparticles could find use in 3-terminal gated devices which can be used directly as switches, amplifiers or logic gates. By linking the metal particles with organic semiconductors, it will be possible to develop 2-terminal switching devices, showing, for example, negative differential resistance (e.g. Fan et al. (2002) JACS 124, 5550-5560). Other possible applications include point sources for emission in field-emission display devices and as conductive inclusions in conductive coatings.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

General Methods

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials used for the growth and maintenance of bacterial cells were obtained from Aldrich Chemicals (Milwaukee, Wis.), DIFCO Laboratories (Detroit, Ml), GIBCO/BRL (Gaithersburg, Md.), or Sigma Chemical Company (St. Louis, Mo.) unless otherwise specified.

The meaning of abbreviations is as follows: “h” means hour(s), “min” means minute(s), “sec” means second(s), “d” means day(s), “mL” means milliliters, “L” means liters.

### Example 1

### Cloning of a Bifunctional Protein

Published Zif268 amino acid sequence (Pavletich NP, Pabo Colo.; Science May 10, 1991; 252 (5007):809-17) was used to generate a DNA sequence using codons most frequently found in E. coli. Using this sequence, a set of 18 oligos (each 31 nucleotide long) was designed to cover the entire gene, and purchased as the 5′ phosphorylated form from Life Technologies (Rockville, Md.). Annealing and ligation reactions were done according to the standard procedure (Sambrook, J., et al, Molecular Cloning: A Laboratory Manual, 2nd Ed., Cold Spring Harbor Laboratory Press, Cold Spring Harbor (1989)). The product was PCR amplified with two primers (#821, #897) that contain BamHI and EcoRI site. After restriction digestion, the PCR product was cloned into pET41A (Novagen, Madison, Wis.) to yield pED105 containing GST-Zif268 fusion protein coding sequence. The cloning vector pET41a contains GST sequence as well as multiple cloning sites. A stop codon TAA was introduced at the end of Zif 268 in pED105 by using Stratagene's “Quick Change” kit (Stratagene, La Jolla, Calif.) to yield pED107. The integrity of all clones was verified by direct sequencing. The sequences are shown below.

Amino acid sequence of Zif268 protein(90 amino acids, 10.8 kDa):

[SEQ ID NO: 28]

MERPYACPVESCDRRFSRSDELTRHIRIHTGQKPFQCRICMRNFSRSDH
LTTHIRTHTGEKPFACDICGRKFARSDERKR
HTKIHLRQKD

Nucleotide sequence of Zif 268 (Converted using E. coli codon):

[SEQ ID NO: 29]

ATGGAACGTCCGTACGCTTGCCCGGTTGAATCTTGCGACCGTCGTTT
CTCTCGTTCTGACGAACTGACCCGTCACATCCG
TATCCACACCGGTCAGAAACCGTTCCAGTGCCGTATCTGCATGCGTA
ACTTCTCTCGTTCTGACCACCTGACCACCCACA
TCCGTACCCACACCGGTGAAAAACCGTTCGCTTGCGACATCTGCGGT
CGTAAATTCGCTCGTTCTGACGAACGTAAACGT
CACACCAAAATCCACCTGCGTCAGAAAGAC

Primers used for Zif268 cloning:

| | |
|---|---|
| 821-ZNF1: | |
| GGGGGATCCATGGAACGTCCGTACGCTTGCC | [SEQ ID NO:30] |
| 822-ZNF2: | |
| CGGTTGAATCTTGCGACCGTCGTTTCTCTCG | [SEQ ID NO:31] |
| 823-ZNF3: | |
| TTCTGACGAACTGACCCGTCACATCCGTATC | [SEQ ID NO:32] |
| 824-ZNF4: | |
| CACACCGGTCAGAAACCGTTCCAGTGCCGTA | [SEQ ID NO:33] |
| 825-ZNF5: | |
| TCTGCATGCGTAACTTCTCTCGTTCTGACCA | [SEQ ID NO:34] |
| 826-ZNF6: | |
| CCTGACCACCCACATCCGTACCCACACCGGT | [SEQ ID NO:35] |
| 827-ZNF7: | |
| GAAAAACCGTTCGCTTGCGACATCTGCGGTC | [SEQ ID NO:36] |
| 828-ZNF8: | |
| GTAAATTCGCTCGTTCTGACGAACGTAAACG | [SEQ ID NO:37] |
| 829-ZNF9: | |
| TCACACCAAAATCCACCTGCGTCAGAAAGAC | [SEQ ID NO:38] |
| 848-ZNR10: | |
| ACCACCACCGTCTTTCTGACGCAGGTGGATT | [SEQ ID NO:39] |
| 849-ZNR11: | |
| TTGGTGTGACGTTTACGTTCGTCAGAACGAG | [SEQ ID NO:40] |
| 850-ZNR12: | |
| CGAATTTACGACCGCAGATGTCGCAAGCGAA | [SEQ ID NO:41] |
| 851-ZNR13: | |
| CGGTTTTTCACCGGTGTGGGTACGGATGTGG | [SEQ ID NO:42] |
| 852-ZNR14: | |
| GTGGTCAGGTGGTCAGAACGAGAGAAGTTAC | [SEQ ID NO:43] |
| 853-ZNR15: | |
| GCATGCAGATACGGCACTGGAACGGTTTCTG | [SEQ ID NO:44] |
| 854-ZNR16: | |
| ACCGGTGTGGATACGGATGTGACGGGTCAGT | [SEQ ID NO:45] |
| 855-ZNR17: | |
| TCGTCAGAACGAGAGAAACGACGGTCGCAAG | [SEQ ID NO:46] |
| 856-ZNR18: | |
| ATTCAACCGGGCAAGCGTACGGACGTTCCAT | [SEQ ID NO:47] |
| PCR primers: | |
| 821-ZNF1: | |
| GGGGGATCCATGGAACGTCCGTACGCTTGCC | [SEQ ID NO:48] |
| 897-2ZnRE: | |
| CCC GAA TTC GTC TTT CTG ACG | [SEQ ID NO:49] |

### Example 2

### Overexpression and Purification of the Bifunctional Proteins

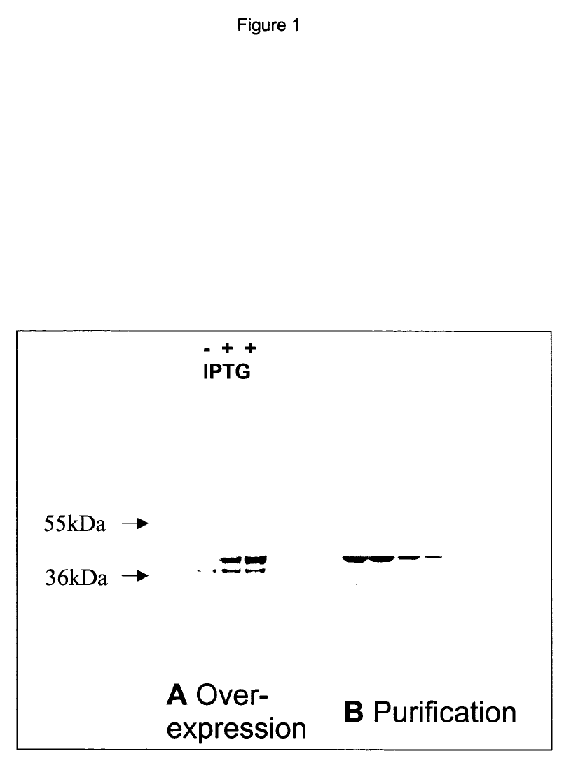

The plasmid pED107 containing GST-Zif268 fusion protein gene was transformed into BL21Star(DE3) (Novagen, Madison Wis.). Overexpression was done by following protocols provided by Novagen. Purification was first done with a GST affinity purification kit (Novagen) and the results are shown in FIG. 1. FIG. 1 is a SDS-PAGE gel (15%) showing induction of the protein at time 0, 1 and 2 hr (lane 2,3 and 4, respectively) after 1 mM IPTG addition. The first lane is protein size marker (Mark 12 from Invitrogen, Carlsbad, Calif.). B shows elution fractions 2-6 in a GST affinity purification experiment.

The calculated molecular weight of GST-Zif268 fusion protein encoded by pED107 was 43 kDa, which is consistent with the observed value for the over-expressed protein.

### Example 3

### Cloning of DNA Fragments Recognized by Zif268

For protein activity assays, a series of plasmid constructs were made which harbor a tandem array of Zif 268 DNA binding sites with copy number ranging from 1 to 23. The Zif268 DNA binding site was defined by sequence 5′-GCGTGGGCG-3′[SEQ ID NO:1] In the constructs, the space between two adjacent Zif268 sites was filled with sequence 5′-MGGGTTCA-3′[SEQ ID NO:2] which is the DNA binding site of another zinc finger protein NRE (Kim JS, Pabo Colo.; Proc Natl Acad Sci USA; Mar. 17, 1998; 95(6):2812-7).

Primers used for cloning are listed below. The 872-NZF and 873-NZR primers encode a NRE site followed by a Zif268 site. The ends of the two primers were designed to have protruding ends MG and TTC so that one unit could anneal with another unit to form multiple copies of binding site (See scheme below).

| | | | |
|---|---|---|---|
| 878-CAPB5′ | AAG . . . 872- | AAG . . . 872- | AAG . . . 880-CAPE5′ |
| | NZF | NZF | |
| 879-CAPB3′ | 873- | 873- | 881-CAPE3′ |
| . . . TTC | NZR . . . TTC | NZR . . . TTC | |

The 874-N/ZF and 875-N/ZR primers encode a NRE site followed by a Zif268 site with one nucleotide g spacer in between. The 876-N//ZF and 877-N//ZR primers encode a NRE site followed by a Zif268 site with two nucleotides GT spacer in between. The growth of the binding site unit is stopped by two pairs of capping primers on the two ends. The capping primers also incorporate BamHI and EcoRI site.

Annealing was done in 0.1 M NaCI, followed by a ligation reaction with T4 ligase. Then, the ligation reaction mixture was used as template for PCR reaction with primer 878 and 881. After cleaning up, the PCR product was digested with BamHI and EcoRI and cloned into pUC18. Clones were sequenced and the number of binding sites in each clone was identified according to the sequencing results. Clones were obtained containing between 1 to 23 Zif268 binding sites in tandem array.

Primers used for the cloning of DNA binding sites:

| | | |
|---|---|---|
| 872-NZF: | AAGGGTTCAGCGTGGGCG | [SEQ ID NO:49] |
| 873-NZR: | CTTCGCCCACGCTGAACC | [SEQ ID NO:50] |
| 874-N/ZF: | AAGGGTTCAGGCGTGGGCGG | [SEQ ID NO:51] |
| 875-N/ZR: | CTTCCGCCCACGCCTGAACC | [SEQ ID NO:52] |
| 876-N//ZF: | AAGGGTTCAGTGCGTGGGCGGT | [SEQ ID NO:53] |
| 877-N//ZR: | CTTACCGCCCACGCACTGAACC | [SEQ ID NO:54] |
| 878-CAPB5′: | GATAAGGATCCGAGCTCG | [SEQ ID NO:55] |
| 879-CAPB3′: | CTTCGAGCTCGGATCCTTATC | [SEQ ID NO:56] |
| 880-CAPE5′: | AAGATGGAATTCGAAGCTTGA | [SEQ ID NO:57] |
| 881-CAPE3′: | TCAAGCTTCGAATTCCAT | [SEQ ID NO:58] |

### Example 4

### Synthesis of GSH Monolayer-Protected Gold Nanoparticles with Average Diameter of 3.0 nm

In a typical reaction, 60 mL MeOH (HPLC grade) and 10 mL acetic acid (HPLC grade) were mixed in a 250 mL Erlenmeyer flask by stirring for 2-5 minutes. 0.37 g (1.0 mmol) tetrachloroauric acid (HAuCl₄.x H₂O) (99.99%) and 61.4 mg (0.2 mmol) glutathion (GSH, minimum 99% from Sigma) were added to the above mixed solvents and dissolved by stirring for 5 minutes, resulting in a clear and yellow solution. Next, 0.6 g (16 mmol) sodium borohydride (NaBH₄, 99%) was dissolved in 30 g Nanopure® water. The NaBH₄ solution was dropwise added into the above solution with rapid stirring. With the first drop of NaBH₄ was added, the HAuCl₄ solution was immediately turned to dark brown from yellow. The reaction was exothermic; the heat generated in the reaction kept the solution warm for ˜15 min. During the reaction, the pH of the solution changed from 1.2 to ˜5.0. Stirring was continued for two hours. The resulting GSH protected gold nanoparticles were soluble in water. When diluted, the solution became purple and clear.

### Example 5

### DNA Binding Activity Assay

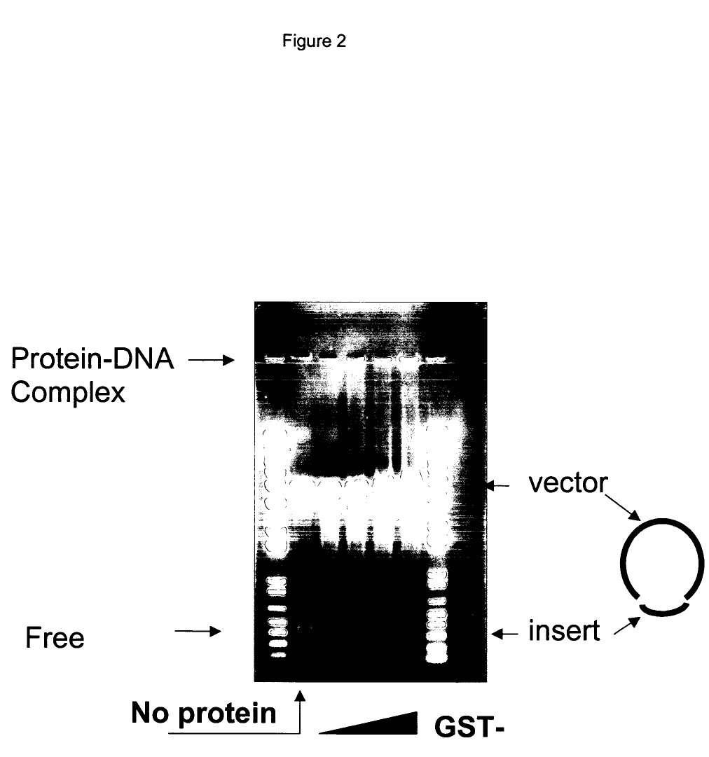

Gel electrophoresis was used to verify that the purified Zn finger protein was bound to the target DNA sequences with high affinity and specificity as shown in FIG. 2. FIG. 2 is a gel retardation assay. Lanes 1 and 7 are “1 Kb Plus DNA ladder” from Life Technologies (Rockville, Md.). Lanes 2-6 show separations from binding reactions of same amount of DNA with increasing amount of proteins as described below.

In this example, two different pieces of DNA's were mixed with PED107 (GST-Zif268 fusion protein); the shorter piece containing 23 Zif268 binding sites, and the longer piece containing no binding sites. DNA was prepared as following: 5 μg (˜2 pmole) of plasmid NZ-23 was digested with BamHI and EcoRI to obtain a 420 bp fragment that contains 23 NZ binding sites and a longer piece corresponding to linearized pUC18 vector. After purification, the DNA was equally divided into five tubes containing 0, 1, 2, 3 and 4 μL of 1 mg/mL (˜20 pmol/μL) PED107. Total reaction volume was brought up to 10 μL with H₂O. The reaction mixture was incubated at RT for 10 min and loaded onto 1% agarose gel (lane 2-5 in FIG. 2). Samples were electrophorized for 60 min at 90V. As the protein concentration was increased, the shorter DNA piece, but not the longer piece, was trapped in the gel well, indicating specific complex formation between Zn finger protein and its target sequence.

### Example 6

### Nanoparticle Binding Activity Assay

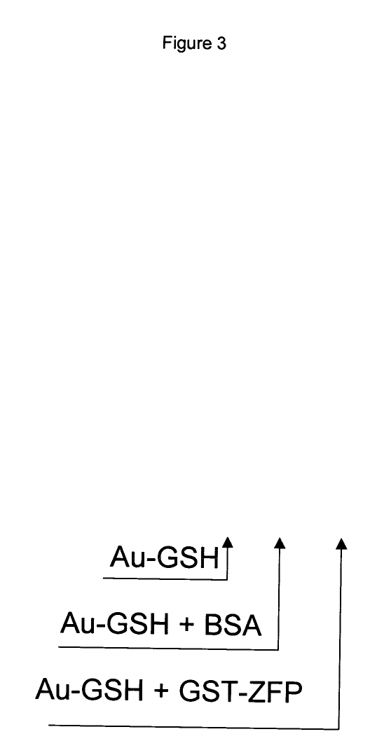

In a typical experiment, 2 μL of Au-GSH particle at a concentration equivalent to OD500=10 (˜10 μM) was mixed with 8 μL of 1 mg/mL GST-Zif268 protein or BSA (Sigma, St. Louis, Mo.) as a control (˜20 μM). After incubation at room temperature for 10 min, the entire reaction mixture was loaded on 1% agarose gel. Gel electrophoresis was run in 1X TBE buffer (Tris-borate-EDTA) at 90V constant voltage for 20 min. Gel pictures were taken by directly scanning the gel on a HP ScanJet 6300C. As shown in FIG. 3, BSA protein cannot bind to Au-GSH particles, whereas the bifunctional protein GST-Zif268 retards mobility of GSH-Au particles, indicative of complex formation between GST-Zif268 and Au-GSH.

### Example 7

### Bifunctionality Assay

This example tested whether the bifunctional protein PED107 (GST-Zif268) would bind to both Au-GSH particle and DNA sequence harboring Zif268 binding sites.

Three reactions were set up as following: 1) 2 μL of Au-GSH particle at concentration OD500=10 (˜10 μM); 2) 2 μL of Au-GSH particle at concentration OD500=10 (˜10 μM) was mixed with 8 μL of 1 mg/mL GST-Zif268 protein; and 3) same as 2, but with 5 μL of NZ-23 (1 μg/μL) added.

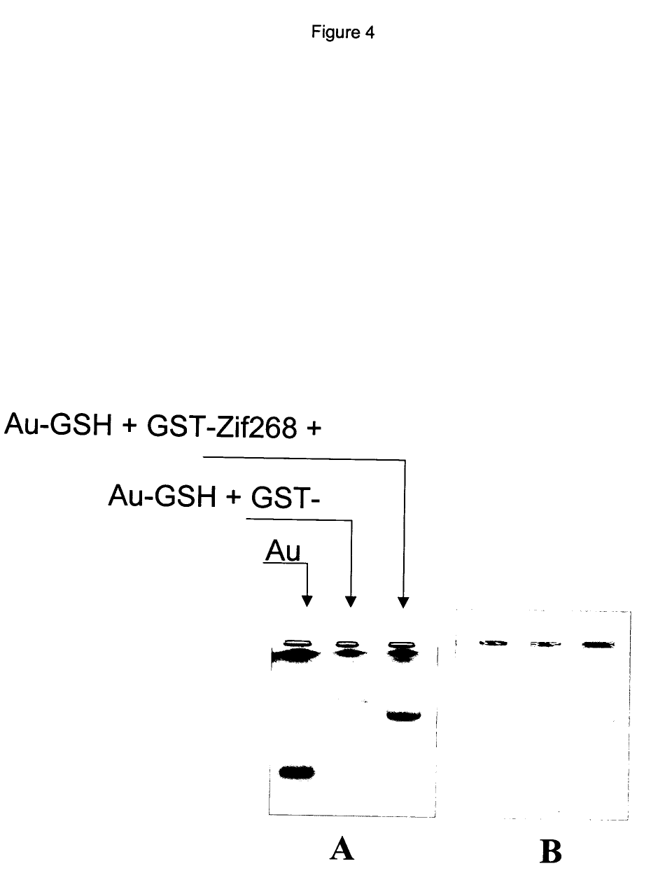

After incubation at room temperature for 10 min, the entire reaction mixture was loaded on 1% agarose gel. Gel electrophoresis was run in 1×TAE buffer (Tris-acetate-EDTA) at 90V constant voltage for 20 min. Gel pictures were taken by directly scanning the gel on a HP ScanJet 6300C. As can be seen in FIG. 4, addition of a piece of DNA that contains protein recognition sequence caused the protein-particle complex to migrate at a different speed, and DNA co-migrated with Au particles. FIG. 4A is the gel image as recorded with the ScanJet 6300C. FIG. 4B is the gel image under UV illumination, recorded with Eagle Eye II (Stratagene, La Jolla, Calif.). These results indicate that the bifunctional protein made in Example 2 can indeed direct a particle onto DNA.

What is claimed is:

1. A functionalized nanoparticle comprising:
a) a nanoparticle coated with a monolayer comprising a capture coating component;
b) a bifunctional protein having a first binding domain and a second binding domain, the first and second binding domains each comprising a member of a binding pair;
wherein the bifunctional protein is affixed to the nanoparticle of (a) at the first binding domain.

2. A functionalized nanoparticle of claim 1 wherein the nanoparticle is metallic.

3. A functionalized nanoparticle according to claim 2 wherein the metal comprising the nanoparticle is selected from the group consisting of gold, silver, platinum, palladium, iridium, rhodium, osmium, iron, copper, cobalt, and alloys thereof.

4. A functionalized nanoparticle of claim 1 wherein the nanoparticle is a semiconductor.

5. A functionalized nanoparticle according to claim 4 wherein the semiconductor comprising the nanoparticle is selected from the group consisting of cadmium selenide, cadmium sulfide, silver sulfide, cadmium sulfide, zinc sulfide, zinc selenide, lead sulfide, gallium arsenide, silicon, tin oxide, iron oxide, and indium phosphide.

6. A functionalized nanoparticle according to claim 1 wherein said monolayer is selected from the group consisting of:
a) molecules having reactive groups selected from the group consisting of: —NH₂, —COOH, —CHO—, —OH, —X (Cl, Br, I), succinimide, and epoxy groups; and
b) biomolecules selected from the group consisting of:
peptides; tiopronin and GSH.

7. A functionalized nanoparticle according to claim 1 wherein said monolayer further comprises a shielding component.

8. A functionalized nanoparticle according to claim 7 wherein the shielding component is selected from the group consisting of: short chain ethylene glycol oligomers, ethylene glycol methacrylate, sugars, crown ethers, and acrylamide.

9. A functionalized nanoparticle according to claim 1 wherein said first binding domain comprises a member of a binding pair selected from the group consisting of: Glutathione-S-transferase/glutathione, 6× Histidine Tag/Ni-NTA, Streptavidin/biotin, S-protein/S-peptide, Cutinase/phosphonate inhibitor, antigen/antibody, hapten/anti-hapten, folic acid/folate binding protein, and protein A or G/immunoglobulins.

10. A functionalized nanoparticle according to claim 1 wherein said second binding domain is a nucleic acid binding amino acid sequence.

11. A functionalized nanoparticle according to claim 10 wherein said nucleic acid binding amino acid sequence is selected from the group consisting of DNA binding domains, and RNA binding domains.

12. A functionalized nanoparticle according to claim 11 wherein said nucleic acid binding amino acid sequence is described by the citations selected from the group consisting of: Genbank accession number: NP_{—}417816, Genbank accession number: P03040, Genbank accession number: NP_{—}040628, Genbank accession number: NP_{—}059642, Genbank accession number: NP_{—}059641.

13. A functionalized nanoparticle according to claim 10 wherein the nucleic acid binding amino acid sequence is a DNA binding zinc finger sequence.

14. A functionalized nanoparticle according to claim 13 wherein the zinc finger sequence binds to a target sequence selected from the group consisting of SEQ ID NO:1, SEQ ID NO:2 and SEQ ID NO:3

15. A functionalized nanoparticle according to claim 1 wherein said second binding domain has affinity for capture moieties selected from the group consisting of nucleic acids, peptides, biological cells, and inorganic nanotubes

16. A functionalized nanoparticle according to claim 15 wherein the second binding domain of the bifunctional peptide has affinity for a carbon nanotube.

17. A functionalized nanoparticle according to claim 16 wherein the bifunctional peptide comprises an amino acid sequence having carbon nanotube binding affinity selected from the group consisting of SEQ ID NO's 4-27.

18. A method for capturing a capture moiety comprising contacting a capture moiety selected from the group consisting of nucleic acids, peptides, biological cells, and inorganic nanotubes with the functionalized nanoparticle of claim 1.

19. A method according to claim 18 wherein the functionalized nanoparticle comprises:
a) a nanoparticle coated with a monolayer comprising a capture coating component;
b) a bifunctional protein having a first binding domain and a second binding domain, the first binding domain comprising a member of a binding pair, the second binding domain comprising a nucleic acid binding amino acid sequence;
wherein the bifunctional protein is affixed to the nanoparticle of (a) through the first binding domain.

20. A method according to claim 19 wherein the capture moiety is a nucleic acid.

21. A method according to claim 19 wherein the nucleic acid binding amino acid sequence is a DNA binding zinc finger sequence.

22. A method according to claim 19 wherein the zinc finger sequence binds to a target sequence selected from the group consisting of SEQ ID NO:1, SEQ ID NO:2 and SEQ ID NO:3.

23. A nucleic acid nanoparticle complex comprising:
a) a nanoparticle coated with a monolayer comprising a capture coating component;
b) a bifunctional protein having a first binding domain and a second binding domain, the first binding domain comprising a member of a binding pair, the second binding domain comprising a nucleic acid binding amino acid sequence;
wherein the bifunctional protein is affixed to the nanoparticle of (a) through the first binding domain and is affixed to a nucleic acid fragment at nucleic acid binding amino acid sequence.

24. A nucleic acid nanoparticle complex according to claim 23 wherein the nucleic acid fragment is immobilized on a solid support.

25. A nucleic acid nanoparticle complex according to claim 23 wherein the nucleic acid binding amino acid sequence is a zinc finger.

26. A process for immobilizing a multiplicity of nanaoparticles on a nucleic acid matrix comprising:
a) providing a multiplicity of functionalized nanoparticles, each comprising:
i) a nanoparticle coated with a monolayer comprising a capture coating component;
ii) a bifunctional protein having a first binding domain and a second binding domain, the first binding domain comprising a member of a binding pair, the second binding domain comprising a nucleic acid binding amino acid sequence;
wherein the bifunctional protein is affixed to the metallic nanoparticle through the first binding domain and wherein the nucleic acid binding amino acid sequence of each second binding domain is unique;
b) providing a nucleic acid matrix having peptide binding domains having affinity for the nucleic acid binding amino acid sequence of each second binding domain of each bifunctional peptide of (a);
c) contacting the functionalized nanoparticles of (a) with the nucleic acid matrix of (b) under conditions whereby the nucleic acid binding amino acid sequence of the bifunctional peptide bind to peptide binding domains of the nucleic acid matrix of (b) to immobilize the nanoparticles.

27. A nanometer scale electronic device made by the process of claim 26.

28. A nanometer scale electronic device of claim 27 selected from the group consisting of an electronic heterojucntion, an electronic interconnect an a nano-wire.

29. A functionalized carbon nanotube comprising:
a) a carbon nanotube; and
b) a bifunctional protein having a first binding domain and a second binding domain, the first binding domain having affinity for a carbon nanotube and the second binding domain comprising a member of a binding pair;
wherein the bifunctional protein is affixed to the carbon nanotube of (a) through the first binding domain.

30. A functionalized carbon nanotube according to claim 29 wherein said second binding domain is a nucleic acid binding amino acid sequence.

31. A functionalized nanoparticle according to claim 30 wherein said nucleic acid binding amino acid sequence is selected from the group consisting of DNA binding domains, and RNA binding domains.

32. A functionalized nanoparticle according to claim 31 wherein said nucleic acid binding amino acid sequence is described by the citations selected from the group consisting of Genbank accession number: NP_{—}417816, Genbank accession number: P03040, Genbank accession number: NP_{—}040628, Genbank accession number: NP_{—}059642, Genbank accession number: NP_{—}059641.

33. A functionalized carbon nanotube according to claim 31 wherein the nucleic acid binding amino acid sequence is a DNA binding zinc finger sequence.

34. A functionalized carbon nanotube according to claim 33 wherein the zinc finger sequence binds to a target sequence selected from the group consisting of SEQ ID NO:1, SEQ ID NO:2 and SEQ ID NO:3.


SEQUENCE LISTING (58 sequences)

SEQ ID NO: 1 - DNA, 9 bp, Artificial Sequence
  Zif268 - zinc finger
gcgtgggcg

SEQ ID NO: 2 - DNA, 9 bp, Artificial Sequence
  NRE - zinc finger
aagggttca

SEQ ID NO: 3 - DNA, 9 bp, Artificial Sequence
  Sp1C zinc finger
ggggcgggg

SEQ ID NO: 4 - protein, 12 aa, phage M10 CNP1
DPHHHWYHMHQH

SEQ ID NO: 5 - protein, 12 aa, phage M10 - CNP2
HAHSQWWHLPYR

SEQ ID NO: 6 - protein, 12 aa, phage M10 - CNP3
HAHSRRGHIQHR

SEQ ID NO: 7 - protein, 12 aa, phage M10 - CNP4
HCHHPWGAWHTL

SEQ ID NO: 8 - protein, 12 aa, phage M10 - CNP5
HCWNQWCSRHQT

SEQ ID NO: 9 - protein, 12 aa, phage M10 - CNP6
HGNWSYWWSKPS

SEQ ID NO: 10 - protein, 12 aa, phage M10 - CNP7
HHWHHWCMPHKT

SEQ ID NO: 11 - protein, 12 aa, phage M10 - CNP8
HNWYHWWMPHNT

SEQ ID NO: 12 - protein, 12 aa, phage M10 - CNP9
HNWYRWCIRHNN

SEQ ID NO: 13 - protein, 12 aa, phage M10 - CNP10
HRWYRWSSRNQT

SEQ ID NO: 14 - protein, 12 aa, phage M10 - CNP11
HSSWWLALAKPT

SEQ ID NO: 15 - protein, 12 aa, phage M10 - CNP12
HWCAWWISSNQS

SEQ ID NO: 16 - protein, 12 aa, phage M10 - CNP13
HWKHPWGAWDTL

SEQ ID NO: 17 - protein, 12 aa, phage M10 - CNP14
HWSAWWIRSNQS

SEQ ID NO: 18 - protein, 12 aa, phage M10 - CNP15
HWSPWHRPWYQP

SEQ ID NO: 19 - protein, 12 aa, phage M10 - CNP16
HYSWYSTWWPPV

SEQ ID NO: 20 - protein, 12 aa, phage M10 - CNP17
HYWWRWWMPNQT

SEQ ID NO: 21 - protein, 12 aa, phage M10 - CNP18
KCHSRHDHIHHH

SEQ ID NO: 22 - protein, 12 aa, phage M10 - CNP19
KSLSRHDHIHHH

SEQ ID NO: 23 - protein, 12 aa, phage M10 - CNP20
KSRSRHDEIHHH

SEQ ID NO: 24 - protein, 12 aa, phage M10 - CNP21
KYRSRHDHIHHH

SEQ ID NO: 25 - protein, 12 aa, phage M10 - CNP22
QWHSRHDHIHHH

SEQ ID NO: 26 - protein, 12 aa, phage M10 - CNP23
HHHHLRHPFWTH

SEQ ID NO: 27 - protein, 12 aa, phage M10 - CNP24
WPHHPHAAHTIR

SEQ ID NO: 28 - protein, 90 aa, mouse zinc finger sequence Zif268p
MERPYACPVESCDRRFSRSDELTRHIRIHTGQKPFQCRICMRNFSRSDHLTTHIRTHTGEKPFACDICGRKFARSDERKRHTKIHLRQKD

SEQ ID NO: 29 - DNA, 270 bp, Artificial Sequence
  Zif268full codons for zinc finger recognition
atggaacgtccgtacgcttgcccggttgaatcttgcgaccgtcgtttctctcgttctgacgaactgacccgtcacatccgtatccacaccggtcagaaaccgttccagtgccgtatctgcatgcgtaacttctctcgttctgaccacctgaccacccacatccgtacccacaccggtgaaaaaccgttcgcttgcgacatctgcggtcgtaaattcgctcgttctgacgaacgtaaacgtcacaccaaaatccacctgcgtcagaaagac

SEQ ID NO: 30 - DNA, 31 bp, Artificial Sequence
  821-ZNF1 primer
gggggatccatggaacgtccgtacgcttgcc

SEQ ID NO: 31 - DNA, 31 bp, Artificial sequence
  822-ZNF2 primer
cggttgaatcttgcgaccgtcgtttctctcg

SEQ ID NO: 32 - DNA, 31 bp, Artificial sequence
  823-ZNF3 PRIMER
ttctgacgaactgacccgtcacatccgtatc

SEQ ID NO: 33 - DNA, 31 bp, Artificial sequence
  824-ZNF4 PRIMER
cacaccggtcagaaaccgttccagtgccgta

SEQ ID NO: 34 - DNA, 31 bp, Artificial sequence
  825-ZNF5 PRIMER
tctgcatgcgtaacttctctcgttctgacca

SEQ ID NO: 35 - DNA, 31 bp, Artificial sequence
  826-ZNF6 PRIMER
cctgaccacccacatccgtacccacaccggt

SEQ ID NO: 36 - DNA, 31 bp, Artificial sequence
  827-ZNF7 PRIMER
gaaaaaccgttcgcttgcgacatctgcggtc

SEQ ID NO: 37 - DNA, 31 bp, Artificial sequence
  828-ZNF8 primer
gtaaattcgctcgttctgacgaacgtaaacg

SEQ ID NO: 38 - DNA, 31 bp, Artificial sequence
  829-ZNF9 primer
tcacaccaaaatccacctgcgtcagaaagac

SEQ ID NO: 39 - DNA, 31 bp, Artificial sequence
  848-ZNR10 primer
accaccaccgtctttctgacgcaggtggatt

SEQ ID NO: 40 - DNA, 31 bp, Artificial sequence
  849-ZNR11  primer
ttggtgtgacgtttacgttcgtcagaacgag

SEQ ID NO: 41 - DNA, 31 bp, Artificial sequence
  850-ZNR12 primer
cgaatttacgaccgcagatgtcgcaagcgaa

SEQ ID NO: 42 - DNA, 31 bp, Artificial sequence
  851-ZNR13 primer
cggtttttcaccggtgtgggtacggatgtgg

SEQ ID NO: 43 - DNA, 31 bp, Artificial sequence
  852ZNR14 primer
gtggtcaggtggtcagaacgagagaagttac

SEQ ID NO: 44 - DNA, 31 bp, Artificial sequence
  853-ZNR15 primer
gcatgcagatacggcactggaacggtttctg

SEQ ID NO: 45 - DNA, 31 bp, Artificial sequence
  854-ZNR16 primer
accggtgtggatacggatgtgacgggtcagt

SEQ ID NO: 46 - DNA, 31 bp, Artificial sequence
  855-ZNR17 primer
tcgtcagaacgagagaaacgacggtcgcaag

SEQ ID NO: 47 - DNA, 31 bp, Artificial sequence
  856-ZNR18 PRIMER
attcaaccgggcaagcgtacggacgttccat

SEQ ID NO: 48 - DNA, 21 bp, Artificial sequence
  897-2ZnRE primer
cccgaattcgtctttctgacg

SEQ ID NO: 49 - DNA, 18 bp, Artificial sequence
  872-NZF PRIMER
aagggttcagcgtgggcg

SEQ ID NO: 50 - DNA, 18 bp, Artificial sequence
  873-NZR PRIMER
cttcgcccacgctgaacc

SEQ ID NO: 51 - DNA, 20 bp, Artificial sequence
  874-N/ZF PRIMER
aagggttcaggcgtgggcgg

SEQ ID NO: 52 - DNA, 20 bp, Artificial sequence
  875-N/ZR PRIMER
cttccgcccacgcctgaacc

SEQ ID NO: 53 - DNA, 22 bp, Artificial sequence
  876-N//ZF PRIMER
aagggttcagtgcgtgggcggt

SEQ ID NO: 54 - DNA, 22 bp, Artificial sequence
  877-N//ZR primer
cttaccgcccacgcactgaacc

SEQ ID NO: 55 - DNA, 18 bp, Artificial sequence
  CAPB5′ primer
gataaggatccgagctcg

SEQ ID NO: 56 - DNA, 21 bp, Artificial sequence
  CAPB3′ primer
cttcgagctcggatccttatc

SEQ ID NO: 57 - DNA, 21 bp, Artificial sequence
  CAPE5′ primer
aagatggaattcgaagcttga

SEQ ID NO: 58 - DNA, 18 bp, Artificial sequence
  CAPE3′ primer
tcaagcttcgaattccat